(19) United States
(12) Patent Application Publication
(10) Pub. No.: US 2016/0346356 A1
(43) Pub. Date: Dec. 1, 2016

(54) USE OF RNA INTERFERENCE, FARNESOIC ACID AND RECOMBINANT PROTEIN APPROACH TO INDUCE GONAD MATURATION IN CRUSTACEAN SPECIES

(71) Applicant: Siu Ming Chan, Zhanjiang (CN)

(72) Inventor: Siu Ming Chan, Zhanjiang (CN)

(21) Appl. No.: 15/236,362

(22) Filed: Aug. 12, 2016

Related U.S. Application Data
(63) Continuation of application No. 14/506,765, filed on Oct. 6, 2014.
(60) Provisional application No. 61/887,323, filed on Oct. 4, 2013.

(51) Int. Cl. A61K 38/22; A61K 38/17; A61K 31/713

(52) U.S. Cl. CPC A61K 38/22; A61K 31/713; A61K 38/1767

(57) ABSTRACT

This invention is directed to the compositions and methods for inducing female gonad maturation in crustacean species such as shrimp, lobster or crab. In one embodiment, the composition for inducing gonad maturation comprises a dsRNA corresponding to the GIH DNA, recombinant protein of a gonad stimulatory hormone (GSH) and farnesoic acid (FA). In another embodiment, the composition for inducing gonad maturation comprises a dsRNA corresponding to the GIH DNA, recombinant MeMIH-B protein and farnesoic acid (FA).

### CROSS-REFERENCE TO RELATED APPLICATION

This application claims priority of U.S. Ser. No. 61/887,323, filed Oct. 4, 2014. The content of the preceding application is hereby incorporated in its entirety by reference into this application.

### FIELD OF THE INVENTION

The present invention relates to compositions and methods for inducing gonad maturation in crustacean species such as shrimp, lobster or crab.

### BACKGROUND OF THE INVENTION

Female reproduction in crustaceans is controlled by an elaborate endocrine system. The prominent cellular activity that occurs during ovarian development is known as vitellogenesis, which is the process whereby vitellogenin (Vg), a yolk protein precursor, is accumulated in the developing oocyte [1]. Vitellogenesis is an essential step in ovarian maturation. Vg can be synthesized in the ovary and/or other nonovarian sites such as the hepatopancreas [2-5]. Vg synthesis and ovarian maturation are regulated by an eyestalk endocrine factor referred to as vitellogenesis-inhibiting hormone (VIH) or gonad-inhibiting hormone (GIH) [6, 7]. Gonad-inhibiting hormone is a member of the neuropeptide family that is synthesized in neuroendocrine cells located in the eyestalk medulla terminalis ganglionic X-organ. Once produced, these neuropeptides are transported to the axon terminals that form a neurohaemal organ called the sinus gland, from where they are secreted [8].

This hormone family is known as the CHH family. These neuropeptides can regulate a variety of physiologic processes, including molting, carbohydrate metabolism, and reproduction. Mature peptides of CHH family members generally have 78-83 amino acid residues with a molecular mass of 8-9 kDa. These hormones contain six cysteine residues that are aligned in conserved positions [9, 10]. The CHH family can be divided into two types, type I and type II, as reflected by their primary structure [11-13]. The most abundant hormone in this family, crustacean hyperglycemic hormone (CHH), belongs to type I, whereas the other two hormones, molt-inhibiting hormone (MIH) and gonad-inhibiting hormone (GIH), are categorized in type II. CHH or type I contains in its precursor sequence a short peptide called CHH-precursor-related peptide followed by a dibasic residue processing site. By contrast, type II hormones are preceded directly by the signal peptides. In addition, alignment of the amino acid sequence reveals deletion of the amino acid glycine at the fifth position after the first cysteine residue in type I peptides.

Compared with CHH and MIH, only a limited number of GIH have been characterized to date. The first peptide with in vivo GIH activity was isolated from the American lobster Homarus americanus [14]. Although the precise mechanism is not known, GIH is postulated to inhibit reproduction by suppressing ovary growth or vitellogenesis [9, 15]. Eyestalk ablation removes the source of GIH and results in ovary growth. In contrast, when eyestalk-ablated females were injected with eyestalk extract, the gonad stimulatory effect of eyestalk ablation was abolished. In addition to GIH, a factor found in the brain and thoracic ganglion of decapod has been implicated in the stimulation of gonad maturation. Injection of protein extract from thoracic ganglion or the brain can stimulate gonad maturation [16].

In sand shrimp Metapenaeus ensis, two forms of MIH-like cDNA (i.e. MeMIH-A and MeMIH-B) have been cloned and characterized [17,18]. MeMIH-B shows only 68% amino acid similarity to MeMIH-A, and amino acid sequence alignment indicates that MeMIH-B is more closely related to GIH of the lobster Homarus americanus [15] than to the mandibular organ-inhibiting hormones of the crab Cancer pagarus [19]. MeMIH-A and MeMIH-B are non-sex-specific and are expressed in the eyestalks of males and females. The expression of MeMIH-A is molt-stage-related, whereas the expression of MeMIH-B is correlated with the reproductive cycle. In addition to the eyestalk, MeMIH-B is also expressed in the brain. MeMIH-B transcript level is low in the initial phase of gonad maturation and increases towards the end of maturation [17]. These findings suggest that the two neuropeptides should have different functions. As they share relatively high sequence similarity, cross-bioactivity also occurs for these two neuropeptides [17]. For example, injection of recombinant MeMIH-B also delays the process of molting [17, 20]. At the time when MeMIH-B was characterized, only a few CHH type II neuropeptides were reported [15, 13]. Despite its potential involvement in reproduction, no further research on the reproductive function of MeMIH-B has been attempted, as there is a lack of a good bioassay system for the neuropeptide. The recent cloning and characterization of the gene encoding the major yolk protein, vitellogenin, may provide a potential biomarker to elucidate the hormonal control of female reproduction [17].

RNA interference (RNAi) technique has been used to define the biological function of many genes. This technique is based on the gene-silencing effect of dsRNA [22]. The technique has revolutionized ‘reverse genetic’ research by introducing dsRNA to organisms or cells. dsRNA can knock down a gene and will produce a phenotypic loss of function of that gene. Although the complete mechanism has yet to be revealed, successful RNAi has been reported for many animal models. For example, Caenorhabditis elegans can be soaked in dsRNA or can be fed plasmids that make dsRNA and consequently exhibit RNAi effects. In many studies, dsRNA can move across cell boundaries freely. Thus, it is not necessary to inject dsRNA directly into the gonad to get progeny that exhibit RNAi effects [22]. As RNAi works in many organisms, it might also work in shrimp. Gene function analysis by RNAi may be advantageous as compared to other conventional approaches.

A cDNA encoding GIH from Penaeus monodon (Pem-GIH) and its potential role in vitellogenesis were studied [23]. Double-stranded RNA, corresponding to the mature Pem-GIH sequence, can trigger a decrease in Pem-GIH transcript levels both in eyestalk ganglia and abdominal nerve cord explant culture and in female P. monodon broodstock. The conspicuous increase in Vg transcript level in the ovary of GIH-knockdown shrimp suggests a negative influence for Pem-GIH on Vg gene expression, and thus implies its role as a gonad-inhibiting hormone [23].

Role of MeMIH-B on regulating vitellogenesis in Metapenaeus ensis was also investigated similarly by RNAi technique [24]. Injection of MeMIH-B dsRNA into the female shrimp caused a decrease in MeMIH-B transcript level in thoracic ganglion and eyestalk. These shrimp also showed reduction of vitellogenin gene expression in the hepatopancreas and ovary. Furthermore, the hemolymph vitellogenin level was also reduced in these animals [24]. Moreover, when hepatopancreas and ovary explants were cultured in medium containing recombinant MeMIH-B, the vitellogenin gene (MeVg1) expression level was upregulated in a dose-dependent manner, reaching a maximum in explants treated with 0.3 μM recombinant MeMIH-B [24].

Several hormones including farnesoic acid (FA), methyl farnesoate (MF) and 20-hydroxyecdysone (20E)) were reported to be important in regulating Vg gene expression in Lobster [21]. Effect of farnesoic acid (FA) and 20-hydroxyecdysone (20E) on production of vitellogenin by hepatopancreas (HaVg1) was investigated by in vitro organ explant. HaVg1 gene expression was stimulated by FA or 20E in a dose-dependent manner [22]. A 2-fold and 2.2-fold increase in HaVg1 gene expression was observed with 4.2 μM FA and 0.7 μM 20E, respectively. The stimulatory effect by either FA or 20E was observed principally during the first 90 min. Stimulation of HaVg1 gene expression by FA and 20E together is greater (3.3-fold increase) than that of either hormone alone [25].

FA could also stimulate the expression of vitellogenin in red crab (Crarybdis feriatus). Use of FA was demonstrated to be advantageous over use of MF or juvenile hormone since a low level of FA could produce similar effect [26, 27].

Since eyestalk produces the gonad inhibiting hormone (GIH), people use unilateral eyestalk ablation to induce gonad (ovary) maturation of the female shrimp. Removing one eyestalk remove half of the inhibiting factor produced by the eye and cause gonad maturation of the female. However, as the X-organ sinus gland and eyestalk also produce other hormones, eyestalk ablation also affects the level of other hormones produced in the eyestalk and causes physiological stress in the female. The whole physiology of the shrimp would be changed even though she can develop the gonad. Consequently, the eggs produced are of inferior quality and the shrimp larvae have low survival rate and lower resistance to diseases due to depression of immune system. The shrimp larvae may suffer from weakness and poor health in later stage of development. Therefore, alternative approaches for inducing gonad maturation other than eyestalk ablation are needed.

In previous studies, the animals were treated at the intermolt stage [23] or the studies were conducted in vitro [24-27]. In contrast, the present invention provides alternative approaches for inducing female gonad maturation which attains higher efficiency in gonad maturation. The methods described herein only direct to the gene and the hormone involved and therefore impose less harmful impact on the animals.

### SUMMARY OF THE INVENTION

The present invention provides compositions and methods for inducing female gonad maturation in crustacean species such as shrimp, lobster or crab.

In one embodiment, there is provided a novel method for inducing gonad maturation by injecting a composition comprising a doubles-stranded RNA (dsRNA) for GIH DNA, a recombinant protein of gonad stimulatory hormone (GSH) and farnesoic acid (FA) to the animal.

In one embodiment, the present invention provides a composition for inducing gonad maturation of shrimp, lobster or crab comprising a dsRNA corresponding to the GIH DNA, recombinant protein of a gonad stimulatory hormone (GSH) and farnesoic acid (FA).

In another embodiment, the composition for inducing gonad maturation of shrimp, lobster or crab comprises a dsRNA corresponding to the GIH DNA, recombinant MeMIH-B protein and farnesoic acid (FA).

In one embodiment, the method for inducing gonad maturation of shrimp, lobster or crab comprises the steps of preparing a composition comprising a dsRNA corresponding to the GIH DNA, recombinant protein of a gonad stimulatory hormone (GSH) and farnesoic acid (FA) and injecting said composition to shrimp, lobster or crab.

In another embodiment, the method for inducing gonad maturation of the shrimp, lobster or crab comprise the steps of preparing a composition comprising a dsRNA corresponding to the GIH DNA, recombinant MeMIH-B protein and farnesoic acid (FA) and injecting said composition to shrimp, lobster or crab.

### DETAILED DESCRIPTION OF THE INVENTION

The present invention provides a composition for inducing female gonad maturation in crustacean species such as shrimp, lobster or crab, said composition comprises one or more of the components selected from: a dsRNA corresponding to the DNA sequence of a gonad inhibiting hormone (GIH), a recombinant protein of a gonad stimulatory hormone (GSH) and farnesoic acid (FA).

In one embodiment, examples for gonad inhibiting hormone (GIH) include vitellogenesis-inhibiting hormone (VIH), gonad-inhibiting hormone (GIH), or other peptides/proteins that can inhibit the expression of vitellogenin or inhibit gonad maturation including, but not limited to, molt-inhibiting hormone (MIH).

In one embodiment, examples for gonad stimulatory hormone (GSH) include gonad stimulatory hormone (GSH), molt-inhibiting hormone type B (i.e. MeMIH-B), or other peptides/proteins that can stimulate the expression of vitellogenin or stimulate gonad maturation including, but not limited to, crustacean hyperglycemic hormone (CHH).

dsRNA for GIH DNA, recombinant GSH and FA were reported respectively to be able to induce vitellogenesis [23-27]. However, current methods usually inject individual dsRNA for GIH DNA, recombinant gonad stimulatory hormone (GSH) or farensoic acid (FA) to the animals during the intermolt stage which may not achieve the optimal effect for inducing gonad maturation. The present invention provides a composition comprising a dsRNA for GIH DNA, a recombinant protein of GSH and farnesoic acid (FA) which is injected to the animal when they are newly molted (i.e. postmolt stage) to achieve a higher efficiency in gonad maturation. The animals are more likely to respond to the treatment at that stage, probably because the dsRNA can fully exhibit its silencing effect before the gene expression has reached a considerable amount and due to the presence of receptors for the signal transduction of GSH and FA at that stage.

In one embodiment, the GIH in said composition is the GIH of shrimp [23].

In one embodiment, the GIH in said composition is the GIH of lobster.

In one embodiment, the GIH in said composition is the GIH of crab.

In one embodiment, the GIH in said composition is the GIH of other crustacean species.

In one embodiment, the recombinant GSH protein in said composition is MeMIH-B protein.

In one embodiment, the recombinant GSH protein in said composition is other GSH protein.

In one embodiment, the recombinant GSH protein described herein is expressed in E. coli.

In another embodiment, the recombinant GSH protein is expressed using other appropriate expression system.

In one embodiment, the GSH protein is a natural protein purified from appropriate organisms such as shrimp, lobster and crab.

In one embodiment, the composition for inducing female gonad maturation of shrimp, lobster or crab comprises a dsRNA corresponding to the GIH DNA sequence, recombinant GSH protein and farnesoic acid (FA).

In one embodiment, the composition for inducing female gonad maturation of shrimp, lobster or crab comprises a dsRNA corresponding to the GIH DNA sequence, recombinant MeMIH-B protein and farnesoic acid (FA).

In one embodiment, the composition for inducing female gonad maturation of shrimp, lobster or crab comprises a dsRNA corresponding to the GIH DNA sequence and recombinant GSH protein.

In one embodiment, the composition for inducing female gonad maturation of shrimp, lobster or crab comprises a dsRNA corresponding to the GIH DNA sequence and recombinant MeMIH-B protein.

In one embodiment, the composition for inducing female gonad maturation of shrimp, lobster or crab comprises a dsRNA corresponding to the GIH DNA sequence and farnesoic acid (FA).

In one embodiment, the composition for inducing female gonad maturation of shrimp, lobster or crab comprises a recombinant GSH protein and farnesoic acid (FA).

In one embodiment, the composition for inducing female gonad maturation of shrimp, lobster or crab comprises a recombinant MeMIH-B protein and farnesoic acid (FA).

The present invention also provides a method for inducing female gonad maturation of shrimp, lobster or crab, said method comprises the following steps:
a) preparing a composition comprising one or more of the components selected from: a dsRNA corresponding to the GIH DNA sequence, a recombinant protein of a gonad stimulatory hormone (GSH) and farnesoic acid (FA); and
b) injecting the composition in step a) to the shrimp, lobster or crab.

In one embodiment, the present invention provides a method for inducing female gonad maturation of shrimp, lobster or crab, said method comprises the following steps:
a) preparing a composition comprising one or more of the components selected from: a dsRNA corresponding to the GIH DNA sequence, one or more recombinant protein of a gonad stimulatory hormone (GSH) and farnesoic acid (FA); and
b) injecting the composition in step a) to the shrimp, lobster or crab.

In one embodiment, the composition is injected to the animal when the animal has just molted (i.e. in the postmolting stage).

In one embodiment, the recombinant GSH protein used for preparing the composition in the method for inducing female gonad maturation is MeMIH-B protein.

The present invention provides a method for inducing female gonad maturation in crustacean, comprising the steps of:
a) preparing a composition comprising a double-stranded RNA (dsRNA) corresponding to the nucleotide sequence of a gonad inhibiting hormone (GIH), a protein of a gonad stimulatory hormone (GSH) and farnesoic acid (FA); and
b) injecting the composition obtained from step a) to the crustacean.

In one embodiment of the method for inducing female gonad maturation in crustacean, wherein the crustacean is a shrimp, lobster or crab.

In one embodiment of the method for inducing female gonad maturation in crustacean, wherein the gonad stimulatory hormone (GSH) is a molt inhibiting hormone type B protein (MeMIH-B).

In one embodiment of the method for inducing female gonad maturation in crustacean, wherein the gonad stimulatory hormone (GSH) is a recombinant protein or purified protein.

In one embodiment of the method for inducing female gonad maturation in crustacean, wherein the composition is injected to the crustacean when the crustacean has just molted or reached the postmolting stage.

In one embodiment of the method for inducing female gonad maturation in crustacean, wherein the composition comprises 0.1-3.0 μg/g of body weight of dsRNA corresponding to the nucleotide sequence of gonad inhibiting hormone (GIH), 1-30 μM of the protein of gonad stimulatory hormone (GSH) and 0.1-1.0 μg/g of body weight of farnesoic acid (FA).

In one embodiment of the method for inducing female gonad maturation in crustacean, wherein the method for inducing female gonad maturation in crustacean, wherein the composition comprises 0.5 μg/g of body weight of dsRNA corresponding to the nucleotide sequence of gonad inhibiting hormone (GIH), 2 μM of the protein of gonad stimulatory hormone (GSH) and 0.5 μg/g of body weight of farnesoic acid (FA).

The present invention provides a method for enhancing the level of vitellogenin protein in a female crustacean, comprising the steps of:
a) preparing a composition comprising a double-stranded RNA (dsRNA) corresponding to the nucleotide sequence of a gonad inhibiting hormone (GIH), a protein of a gonad stimulatory hormone (GSH) and farnesoic acid (FA); and
b) injecting the composition obtained from step a) to the crustacean.

In one embodiment of the method for enhancing the level of vitellogenin protein in a female crustacean, wherein the crustacean is a shrimp, lobster or crab.

In one embodiment of the method for enhancing the level of vitellogenin protein in a female crustacean, wherein the gonad stimulatory hormone (GSH) is a molt inhibiting hormone type B protein (MeMIH-B).

In one embodiment of the method for enhancing the level of vitellogenin protein in a female crustacean, wherein the gonad stimulatory hormone (GSH) is a recombinant protein or purified protein.

In one embodiment of the method for enhancing the level of vitellogenin protein in a female crustacean, wherein the composition is injected to the crustacean when the crustacean has just molted or reached the postmolting stage.

In one embodiment of the method for enhancing the level of vitellogenin protein in a female crustacean, wherein the composition comprises 0.1-3.0 μg/g of body weight of dsRNA corresponding to the nucleotide sequence of gonad inhibiting hormone (GIH), 1-30 μM of the protein of gonad stimulatory hormone (GSH) and 0.1-1.0 μg/g of body weight of farnesoic acid (FA).

In one embodiment of the method for enhancing the level of vitellogenin protein in a female crustacean, wherein the composition comprises 0.5 μg/g of body weight of dsRNA corresponding to the nucleotide sequence of gonad inhibiting hormone (GIH), 2 μM of the protein of gonad stimulatory hormone (GSH) and 0.5 μg/g of body weight of farnesoic acid (FA).

This invention will be better understood by reference to the examples which follow. However, one skilled in the art will readily appreciate that the examples provided are merely for illustrative purposes and are not meant to limit the scope of the invention which is defined by the claims following thereafter.

Throughout this application, it is to be noted that the transitional term “comprising”, which is synonymous with “including”, “containing” or “characterized by”, is inclusive or open-ended, and does not exclude additional, un-recited elements or method steps.

### EXAMPLE 1

RNA interference is used to knock down the gonad inhibiting hormone (GIH) gene. dsRNA for GIH (e.g. 1 μg/g of body weight) can be injected into adult females of 100 g. Injection of this should start when the females have just molted. In one embodiment, the amount to inject is 1 μg dsRNA/g of body weight and the location of injection is the arthropodial membrane of the walking leg.

After injection, the shrimps will be fed with blood worm, scallop and squid as before. After two day, hemolymph is redrawn for analyzing the level of vitellogenin (egg yolk protein). The level of the vitellogenin should be much higher as compared to the no dsRNA injected control.

### EXAMPLE 2

Farnesoic acid (FA) is known to be able to stimulate vitellogenin synthesis in crab, shrimp and lobster. Stock of FA is prepared in ultra-pure mineral oil and female shrimp will be injected with FA.

Fifty female shrimp are injected with FA through the arthropodial membrane (e.g. at 1 μg/g of body weight) 1 day after they molt. After injection, the shrimp is returned to the culture tank for culture. A group of 10 shrimps are randomly selected at a 24-hour interval. 50 μl of hemolymph sample will be taken from the shrimps for analyzing the vitellogenin level by ELISA. On day 6, all the shrimps will be sacrificed and their ovary will be weighted for estimating the gonadosomatic index (GSI), which is the ratio of ovary weight to total body weight. A control group is also setup in which the shrimps are injected with the same volume of mineral oil without any FA. Shrimps injected with FA should have a higher ovary weight to total shrimp weight than the control.

### EXAMPLE 3

It is known that the brain, thoracic ganglion of the shrimp produce a gonad stimulating hormone (GSH) that stimulates gonad maturation. Gene of the gonad stimulating hormone has been cloned in the sand shrimp Metapenaeus ensis. In vivo recombinant protein injection experiment has confirmed that the GSH can stimulate the expression of the vitellogenin gene. cDNA of the GSH gene has been cloned and can be used to express the recombinant protein for GSH.

In one embodiment, recombinant protein of GSH is obtained using a bacteria (E. coli) expression system. After purification using commercial kit, the protein is dissolved in a PBS buffer at an appropriate concentration (e.g. 4 uM) and injected into newly molted shrimps (i.e. <2 days). A group of 10 shrimps are randomly selected at a 24-hour interval. 50 μl of hemolymph sample will be taken from the shrimps for analyzing the vitellogenin level by ELISA. On day 6, all the shrimps will be sacrificed and their ovary will be weighted for estimating the gonadosomatic index (GSI). A control group is also setup in which the shrimps are treated with PBS alone without any recombinant protein.

In one embodiment, the recombinant protein of GSH is MeMIH-B protein. In another embodiment, the recombinant protein of GSH is other GSH protein.

### EXAMPLE 4

To be more effective to stimulate gonad maturation than eyestalk ablation, a combination of dsRNA for GIH and recombinant GSH protein will be injected into newly molted female. dsRNA for GIH and recombinant GSH protein can be mixed in the same PBS buffer at appropriate concentrations. The dsRNA and GSH protein are then mixed with FA in mineral oil and injected to the animal at once. In one embodiment, the concentrations of dsRNA for GIH, recombinant GSH protein and FA are 0.5 μg/g of body weight, 2 μM and 0.5 μg/g of body weight respectively.

In one embodiment, the doses of dsRNA for GIH are 0.1-3.0 μg/g of body weight. In another embodiment, the doses of dsRNA for GIH are 1.0-3.0 μg/g of body weight. In another embodiment, the doses of dsRNA for GIH are 2.0-2.5 μg/g of body weight. In another embodiment, the doses of dsRNA for GIH are 0.5-0.6 μg/g of body weight. In another embodiment, the doses of dsRNA for GIH are 0.1-0.5 μg/g of body weight.

In one embodiment, the doses of the recombinant GSH protein are 1-30 μM. In another embodiment, the doses of the recombinant GSH protein are 10-30 μM. In another embodiment, the doses of the recombinant GSH protein are 15-25 μM. In another embodiment, the doses of the recombinant GSH protein are 5-10 μM. In another embodiment, the doses of the recombinant GSH protein are 1-5 μM. In one embodiment, two or more types of GSH proteins are injected into the animal at a total dosage recited herein.

In one embodiment, the doses of farnesoic acid (FA) are 0.1-1.0 μg/g of body weight. In another embodiment, the doses of farnesoic acid (FA) are 0.3-0.8 μg/g of body weight. In another embodiment, the doses of farnesoic acid (FA) are 0.4-0.7 μg/g of body weight.

After injection, a group of 10 shrimps are randomly selected at a 24-hour interval. 50 μl of hemolymph sample will be taken from the shrimps for analyzing the vitellogenin level by ELISA. On day 6, all the shrimps will be sacrificed and their ovary will be weighted for estimating the gonadosomatic index (GSI). A control group is also setup in which the shrimps are treated with a mixture of PBS and mineral oil.

### EXAMPLE 5

It is possible that a mixture of these active compounds (dsRNA, recombinant protein and/or farnesoic acid) may have an even higher synergetic effect on gonad maturation. A different combination or permutations of these active compounds will be mixed at various concentrations and tested for gonad development response from the females.

### REFERENCES

1. Tsukimura B (2001) Crustacean vitellogenesis: its role in oocyte development. Am Zool 41, 465-476.
2. Tsutsui N, Kawazoe I, Ohira T, Jasmani S, Yang W-J, Wilder M N & Aida K (2000) Molecular characterization of a cDNA encoding vitellogenin and its expression in the hepatopancreas and ovary during vitellogenesis in the Kuruma prawn, Penaeus japonicus. Zool Sci 17, 651-660.
3. Avarre J-C, Michelis R, Tietz A & Lubzens E (2003) Relationship between vitellogenin and vitellin in a marine shrimp (Penaeus semisulcatus) and molecular characterization of vitellogenin complementary DNAs. Biol Reprod 69, 355-364.
4. Tsang W-S, Quackenbush L S, Chow B K C, Tiu S H K, He J G & Chan S-M (2003) Organization of the shrimp vitellogenin gene: evidence of multiple genes and tissue specific expression by the ovary and hepatopancreas. Gene 303, 99-109.
5. Yang F, Xu H T, Dai Z M & Yang W J (2005) Molecular characterization and expression analysis of vitellogenin in the marine crab Portunus trituberculatus. Comp Biochem Physiol B 142, 456-464.
6. Charmantier G, Charmantier-Daures M & Van Herp F (1997) Hormonal regulation of growth and reproduction in crustaceans. In Endocrinology and Reproduction, Recent Advances in Marine Biotechnology, Vol. 1 (Fingerman M, Nagabhushanam R & Thompson M-F, eds), pp. 109-161. Science Publishers, Enfield, N.H.
7. Huberman A (2000) Shrimp endocrinology. A review. Aquaculture 191, 191-208
8. Skinner D M (1985) Molting and regeneration. In The Biology of Crustacea, Integuments, Pigments and Hormonal Processes, Vol. 9 (Bliss D E & Mantel L H eds), pp. 43-146. Academic Press, Orlando, Fla.
9. Keller R (1992) Crustacean neuropeptides: structures, functions and comparative aspects. Experientia 48, 439-448.
10. Ohira T, Katayama H, Tominaga S, Takasuka T, Nakatsuji T, Sonobe T, Aida K & Nagasawa H (2005) Cloning and characterization of a molt-inhibiting hormone-like peptide from the prawn Marsupenaeus japonicus. Peptide 26, 259-268.
11. Lacombe C, Greve P & Martin G (1999) Overview on the sub-grouping of the crustacean hyperglycemic hormone family. Neuropeptides 33, 71-80.
12. Chen S H, Lin C Y & Kuo C M (2005) In silico analysis of crustacean hyperglycemic hormone family. Mar Biotechnol 7, 193-206.
13. Chan S M, Gu P L, Chu K H & Tobe S S (2003) Crustacean neuropeptide genes of the CHH/MIH/GIH family: implications from molecular studies. Gen Comp Endocrinol 134, 214-219.
14. Soyez D, Van Deijnen J E & Martin M (1987) Isolation and characterization of a vitellogenesis-inhibiting factor from the sinus gland of the lobster, Homarus americanus. J Exp Zool 244, 479-484.
15. De Kleijn D P & Van Herp F (1995) Molecular biology of neurohormone precursors in the eyestalk of Crustacea. Comp Biochem Physiol B Biochem Mol Biol 112, 573-579.
16. Fingerman M (1997) Roles of neurotransmitters in regulating reproductive hormone release and gonadal maturation in decapod crustaceans. Invertebr Reprod Dev 31, 47-54.
17. Gu P L, Tobe S S, Chow B K, Chu K H, He J G & Chan S M (2002) Characterization of an additional molt inhibiting hormone-like neuropeptide from the shrimp Metapenaeus ensis. Peptides 23, 1875-1883.
18. Gu P L & Chan S M (1998) Cloning of a cDNA encoding a putative molt-inhibiting hormone from the eyestalk of the sand shrimp Metapenaeus ensis. Mol Mar Biol Biotechnol 7, 214-220.
19. Lu W, Wainwright G, Webster S G, Rees H H & Turner P C (2000) Clustering of mandibular organ-inhibiting hormone and moult-inhibiting hormone genes in the crab, Cancer pagurus, and implications for regulation of expression. Gene 253, 197-07.
20. Gu P L, Chu K H & Chan S M (2001) Bacterial expression of the shrimp molt-inhibiting hormone (MIH): antibody production, immunocytochemical study and biological assay. Cell Tissue Res 303, 129-136.
21. Tiu S H, Hui H L, Tsukimura B, Tobe S S, He J G & Chan S M (2009) Cloning and expression study of the lobster (Homarus americanus) vitellogenin: conservation in gene structure among decapods. Gen. Comp. Endocrinol. 160, 36-46.
22. Fire A, Xu S, Montgomery M K, Kostas S A, Driver S E & Mello C C (1998) Potent and specific genetic interference by double-stranded RNA in Caenorhabditis elegans. Nature 391, 806-811.
23. Treerattrakool S, Panyim S, Chan S M, Withyachumnarnkul B & Udomkit (2008) Molecular characterization of gonad-inhibiting hormone of Penaeus monodon and elucidation of its inhibitory role in vitellogenin expression by RNA interference. FEBS J 275(5), 970-80.
24. Tiu S H & Chan S M (2007) The use of recombinant protein and RNA interference approaches to study the reproductive functions of a gonad-stimulating hormone from the shrimp Metapenaeus ensis. FEBS J 274(17):4385-9.
25. Tiu S H, Chan S M & Tobe S S (2010) The effects of farnesoic acid and 20-hydroxyecdysone on vitellogenin gene expression in the lobster, Homarus americanus, and possible roles in the reproductive process. Gen Comp Endocrinol. 166, 337-345.
26. Chan S M, Mak A S, Choi C L, Ma T H, Hui J H, Tiu S H (2005) Vitellogenesis in the red crab, Charybdis feriatus: contributions from small vitellogenin transcripts (CfVg) and farnesoic acid stimulation of CfVg expression. Ann N Y Acad Sci 1040, 74-9.
27. Mak A S, Choi C L, Tiu S H, Hui J H, He J G, Tobe S S, Chan S M (2005) Vitellogenesis in the red crab Charybdis feriatus: Hepatopancreas-specific expression and farnesoic acid stimulation of vitellogenin gene expression. Mol Reprod Dev. 70(3), 288-300.

What is claimed is:

1. A method for inducing female gonad maturation in crustacean, comprising the steps of:
c) preparing a composition comprising a double-stranded RNA (dsRNA) corresponding to the nucleotide sequence of a gonad inhibiting hormone (GIH), a protein of a gonad stimulatory hormone (GSH) and farnesoic acid (FA); and
d) injecting the composition obtained from step a) to the crustacean.

2. The method of claim 1, wherein the crustacean is a shrimp, lobster or crab.

3. The method of claim 1, wherein the gonad stimulatory hormone (GSH) is a molt inhibiting hormone type B protein (MeMIH-B).

4. The method of claim 1, wherein the gonad stimulatory hormone (GSH) is a recombinant protein or purified protein.

5. The method of claim 1, wherein the composition is injected to the crustacean when the crustacean has just molted or reached the postmolting stage.

6. The method of claim 1, wherein the composition comprises 0.1-3.0 μg/g of body weight of dsRNA corresponding to the nucleotide sequence of gonad inhibiting hormone (GIH), 1-30 μM of the protein of gonad stimulatory hormone (GSH) and 0.1-1.0 μg/g of body weight of farnesoic acid (FA).

7. The method of claim 1, wherein the composition comprises 0.5 μg/g of body weight of dsRNA corresponding to the nucleotide sequence of gonad inhibiting hormone (GIH), 2 μM of the protein of gonad stimulatory hormone (GSH) and 0.5 μg/g of body weight of farnesoic acid (FA).

8. A method for enhancing the level of vitellogenin protein in a female crustacean, comprising the steps of:
c) preparing a composition comprising a double-stranded RNA (dsRNA) corresponding to the nucleotide sequence of a gonad inhibiting hormone (GIH), a protein of a gonad stimulatory hormone (GSH) and farnesoic acid (FA); and
d) injecting the composition obtained from step a) to the crustacean.

9. The method of claim 8, wherein the crustacean is a shrimp, lobster or crab.

10. The method of claim 8, wherein the gonad stimulatory hormone (GSH) is a molt inhibiting hormone type B protein (MeMIH-B).

11. The method of claim 8, wherein the gonad stimulatory hormone (GSH) is a recombinant protein or purified protein.

12. The method of claim 8, wherein the composition is injected to the crustacean when the crustacean has just molted or reached the postmolting stage.

13. The method of claim 8, wherein the composition comprises 0.1-3.0 μg/g of body weight of dsRNA corresponding to the nucleotide sequence of gonad inhibiting hormone (GIH), 1-30 μM of the protein of gonad stimulatory hormone (GSH) and 0.1-1.0 μg/g of body weight of farnesoic acid (FA).

14. The method of claim 8, wherein the composition comprises 0.5 μg/g of body weight of dsRNA corresponding to the nucleotide sequence of gonad inhibiting hormone (GIH), 2 μM of the protein of gonad stimulatory hormone (GSH) and 0.5 μg/g of body weight of farnesoic acid (FA).
